(19) United States
(12) Patent Application Publication
(10) Pub. No.: US 2026/0054049 A1
(43) Pub. Date: Feb. 26, 2026

(54) HEMOSTASIS VALVE

(71) Applicant: Terumo Corporation, Tokyo (JP)

(72) Inventor: Kevin Ngo, Westminster, CA (US)

(73) Assignee: Terumo Corporation, Tokyo (JP)

(21) Appl. No.: 19/099,467

(22) PCT Filed: Jul. 25, 2023
(86) PCT No.: PCT/US2023/070909

§ 371 Date: Jan. 28, 2025

Related U.S. Application Data
(60) Provisional application No. 63/370,058, filed on Aug. 1, 2022.
(60) Provisional application No. 63/481,358, filed on Jan. 24, 2023.

(51) Int. Cl. A61M 39/06

(52) U.S. Cl. CPC A61M 39/0613; A61M 2039/062

(57) ABSTRACT

A hemostasis valve for use with an access device such as a catheter. The hemostasis valve may be connected to a housing to which the catheter may be connected. The hemostasis valve may include a deformable, resilient tubular member having a passageway. The hemostasis valve be adjustable between a first position in which the passageway is sealed and a second position in which the passageway is unsealed. The passageway may be sealed by one or more cams, with the cams being biased towards the sealed position such that, absent active application of force, the cams revert back to an original position to seal the passageway.

### RELATED APPLICATIONS

This application claims benefit of and priority to U.S. Provisional Application Ser. No. 63/481,358 filed Jan. 24, 2023 entitled Hemostasis Valve, and U.S. Provisional Application Ser. No. 63/370,058 filed Aug. 1, 2022 entitled Hemostasis Valve, both of which are hereby incorporated herein by reference in their entireties.

### BACKGROUND OF THE INVENTION

Valves such as hemostasis valves are primarily used to maintain hemostasis (e.g., prevent bleeding and loss of blood pressure) while one or more devices access the vasculature of a patient. For example, hemostasis valves are often included on a proximal end of an introducer outside of the patient's body so that a guidewire, catheter, or similar device can be advanced through and into the vasculature of the patient.

It is typically important during use of such valves that the operator be aware of the state (e.g., opened or closed) of the valve at all times during a procedure. Failure to recognize that the valve is opened, for example, can lead to excess bleed and potentially air entering the vasculature. In that respect, it can be helpful that such valves are easy to operate (e.g., open and close) so as to minimize distractions or interruptions during a medical procedure.

### SUMMARY OF THE INVENTION

The present invention is generally directed to a hemostasis valve.

In some example embodiments, the hemostasis valve is connected to an access device such as an introducer or a catheter.

In some example embodiments, the hemostasis valve is connected to an aspiration device such as an aspiration catheter.

The hemostasis valve may include a tubular member including a passageway, one or more cams for selectively sealing the passageway of the tubular member, and an actuator for adjusting the plurality of cams. Note that while the present specification may refer to a plurality of cams for simplicity, any of the embodiments herein may alternately include only one cam.

In some example embodiments, the tubular member may comprise a gasket.

In some example embodiments, the plurality of cams may be adjustable between a first position in which the plurality of cams deform the tubular member to seal the passageway and a second position in which the plurality of cams release the tubular member to at least partially unseal the passageway.

In some example embodiments, the plurality of cams may be biased towards a closed, contracted, or sealed position.

In some example embodiments, the plurality of cams may comprise a first cam and a second cam, with the first cam not being directly connected to the second cam.

In some example embodiments, the plurality of cams may comprise two pairs of cams.

In some example embodiments, the plurality of cams may be pivotable between the sealed and opened positions.

In some example embodiments, the tubular member may be positioned between a first cam and a second cam.

In some example embodiments, the tubular member may be pinched between a first cam and a second cam when the first and second cams are in the sealed position.

In some example embodiments, four cams may be radially positioned around a circumference of the tubular member.

In some example embodiments, each of the plurality of cams may be aligned along the same radial plane.

In some example embodiments, the plurality of cams may pivot together such that pivoting of one of the plurality of cams causes the remaining of the plurality of cams to also pivot.

In some example embodiments, the plurality of cams may each be individually and separately pivotable.

In some example embodiments, one or more biasing members may be connected to the plurality of cams to bias the cams towards the sealed position.

In some example embodiments, a first biasing member may be connected between a first cam and an actuator, and a second biasing member may be connected between a second cam and the actuator.

In some example embodiments, one or more biasing members may comprise one or more springs, such as coil springs.

In some example embodiments, the actuator may comprise a ring member including a flange for engaging with a plurality of cams.

In some example embodiments, the actuator may be rotatable to adjust the plurality of cams between the sealed and opened positions.

In some example embodiments, each of the plurality of cams may be pivotably connected to a pin so as to pivot about or with the pin.

In some example embodiments, the present invention may include a housing including an internal lumen for connecting to a catheter, with the tubular member being connected to the housing and the passageway of the tubular member being fluidly connected to the internal lumen of the housing.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which:

FIG. 1 is an isometric view of a hemostasis valve and access device according to one embodiment of the present invention.

FIG. 2 is an exploded view of a hemostasis valve according to one embodiment of the present invention.

FIG. 3A is an end view of a hemostasis valve in a closed position according to one embodiment of the present invention.

FIG. 3B is an end view of a hemostasis valve in an opened position according to one embodiment of the present invention.

FIG. 4A is a sectional view of a hemostasis valve in a closed position according to one embodiment of the present invention.

FIG. 4B is a sectional view of a hemostasis valve in an opened position according to one embodiment of the present invention.

FIG. 5 is an end view of a housing according to one embodiment of the present invention.

FIG. 6A is an end view of a hemostasis valve in a closed position according to one embodiment of the present invention.

FIG. 6B is an end view of a hemostasis valve in an opened position according to one embodiment of the present invention.

FIG. 7 is an exploded view of a hemostasis valve according to one embodiment of the present invention.

FIG. 8A is a sectional view of a hemostasis valve in a closed position according to one embodiment of the present invention.

FIG. 8B is a sectional view of a hemostasis valve in an opened position according to one embodiment of the present invention.

FIG. 9A is an end view of a hemostasis valve in a closed position according to one embodiment of the present invention.

FIG. 9B is an end view of a hemostasis valve in an opened position according to one embodiment of the present invention.

### DETAILED DESCRIPTION

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

Hemostasis valves are typically used at proximal openings of devices that access the vasculature of a patient, such as an introducer, a catheter, or a catheter hub. Hemostasis valves allow other devices, such as guidewires, catheters, implant delivery devices, and similar devices, to pass through and close or seal around the perimeter of the device. Hence, excess blood is prevented from escaping from the vasculature and hemostasis is maintained within the patient.

The present invention is generally directed to a hemostasis valve that can be used in connection with any known medical procedure in which hemostasis valves are presently used, such as guidewire access, catheter access, implant delivery access, and aspiration catheter access.

The hemostasis valve of the present invention may include one or more cams, a portion of which moves between at least a first position to compress or provide radial pressure on a tubular gasket and a second position to allow radial expansion of the tubular gasket. Hence, the one or more cams cause the tubular gasket to open or seal closed against itself or around a device positioned therethrough the tubular gasket.

The hemostasis valve may include 1, 2, 3, 4, 5, 6, or more cams. The cams may be biased in their first position (i.e., a sealed position of the valve) via springs, elastic members, or similar components. The cams may be connected to an actuator component accessible from an outside of the valve to allow hand-actuated movement of the cams and thereby open or close/seal the valve.

The cams may be aligned on a radial plane perpendicular to the axis of rotation such that each of the cams is positioned at the same depth within the valve. However, one or more of the cams may be positioned at a different depth than one or more of the remaining cams such that the one or more of the cams are not aligned with the one or more remaining cams on the radial plane perpendicular to the axis of rotation.

Specific example embodiments are described further below. However, it should be understood that any of the features from any of the embodiments can be mixed and matched with each other in any combination. Hence, the present invention should not be restricted to only these embodiments, but any broader combination thereof.

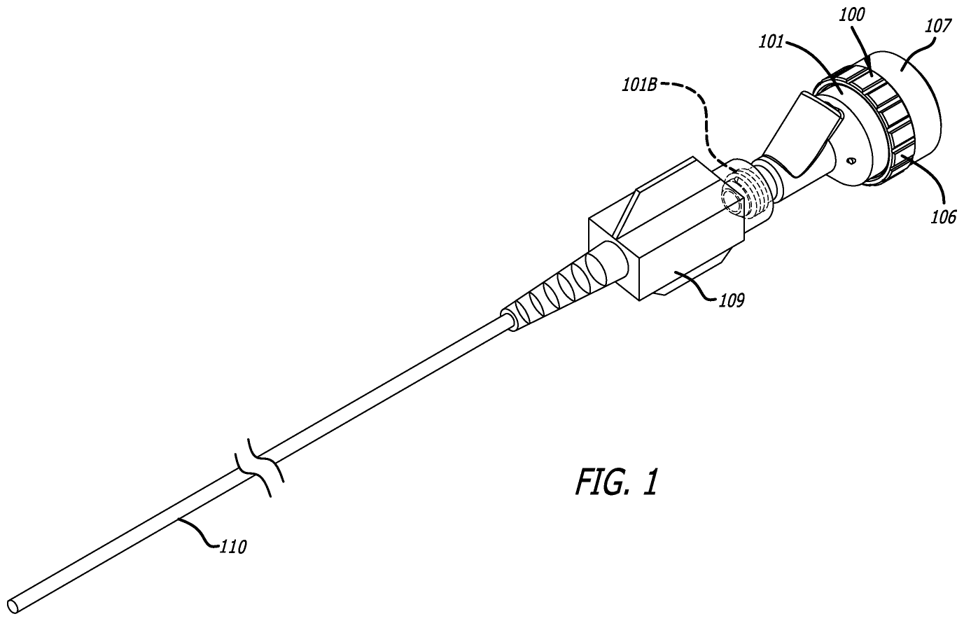

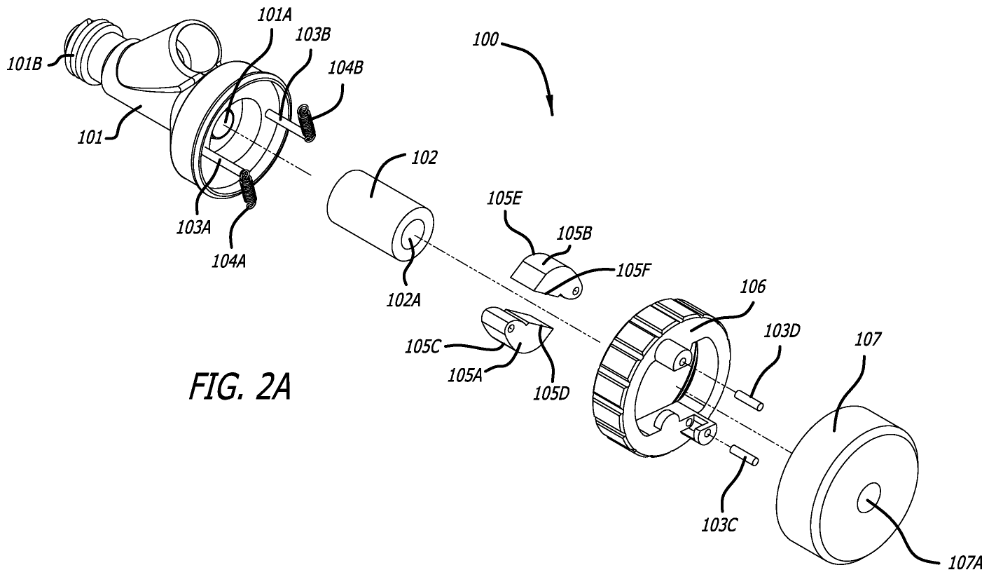

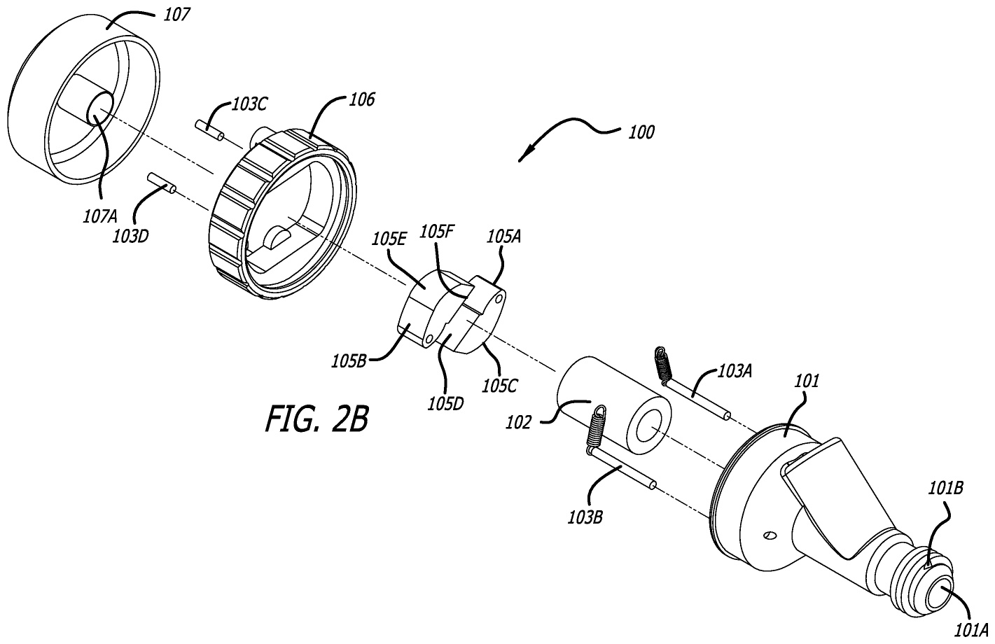

FIG. 1 illustrates one example embodiment of a hemostasis valve 100. In the present example, the hemostasis valve 100 is integrally part of a housing 101 but the valve mechanism itself may alternately take the form of a standalone valve device that directly connects to a further medical device (e.g., the catheter hub of a catheter). Various types of housings 101 may be utilized, and thus the example embodiment illustrated in the figures should not be construed as limiting in scope with respect to, e.g., the shape, size, configuration, number of ports, and the like of the housing 101. The housing 101 may include at least one internal lumen 101A such as shown in FIGS. 2A and 5 through which a medical device may be inserted or removed to/from the housing 101. The housing 101 may include additional lumens in some embodiments. The housing 101 may also include additional ports, each having their own one or more lumens, in some embodiments.

Continuing to reference FIG. 1, it can be seen that an access device such as a catheter 110 may be connected to the housing 101. The access device may be fixed to the housing 101, may be removably attached to the housing 101, or may be integral with the housing 101. The catheter 110 may be removably connected to the housing 101 such that different types of catheters 110 may be interchanged as needed.

In the exemplary embodiment shown in the figures, it can be seen that the housing 101 may include a connector 101B at its distal end to which a catheter 110 or catheter hub 109 may be attached. The connector 101B may comprise threading such that the catheter 110, an adapter, or another connecting structure may be threadably attached thereto. The figures illustrate an embodiment in which the connector 101B includes outer threading (e.g., male threading), and the catheter hub 109 includes inner threading (e.g., female threading). In some embodiments, the opposite configuration may be utilized (e.g., the catheter 110 may include inner threading (e.g., a ring with inner threading) and the catheter hub 109 may include outer threading). It should also be appreciated that, in some embodiments, other types of connectors 101B may be utilized to which a catheter 110, catheter hub 109, or adapter may be attached. In yet other embodiments, as mentioned above, the access device such as a catheter 110 may be integral with the housing 101.

A wide range of access devices may be connected to or integral with the housing 101, and thus the specific configuration of catheter 110 illustrated in the figures should not be construed as limiting in scope. In one example embodiment, an aspiration catheter 110 may be connected to the housing 101, allowing other catheters (e.g., an implant delivery catheter or a drug balloon catheter) to be advanced through the lumen of the valve. In other example embodiments, various types of catheters 110 known in the art such as but not limited to diagnostic catheters, microcatheters, and the like may be connected to the housing 101.

The hemostasis valve 100 may generally be integral with the housing 101 such as shown in FIG. 1. The hemostasis valve 100 may be connected to the housing 101 in an example embodiment. In some embodiments, the hemostasis valve 100 may be fixed to the housing 101 or removably connected to the housing 101. The hemostasis valve 100 may be positioned within the housing 101 as shown in the figures. In such embodiments, the housing 101 may include an internal cavity within which the hemostasis valve 100 is positioned.

FIGS. 2A-2B are exploded views illustrating different components of an example embodiment of a hemostasis valve 100. As shown in FIGS. 2A-2B, the hemostasis valve 100 may comprise a tubular member 102, one or more cams 105A, 105B for selectively sealing a passageway 102A of the tubular member, and an actuator 106 for adjusting the cams 105A, 105B between at least two positions (e.g., a sealed/closed position and an unsealed/opened position).

The shape, size, positioning, orientation, and configuration of the tubular member 102 may vary in different embodiments. In the example embodiment shown in the figures, the tubular member 102 is illustrated as comprising a cylindrical body having an internal passageway 102A. Thus, in the illustrated embodiment, the tubular member 102 may comprise a circular cross-section. However, in other embodiments, the shape of the tubular member 102 may be different than is shown in the exemplary figures. For example, the tubular member 102 may in some embodiments have a triangular-, rectangular-, ovular-, or square-shaped cross-section.

The tubular member 102 may comprise a flexible or semi-rigid member having a passageway 102A extending therethrough. The tubular member 102 may be resilient. In a preferred embodiment, the tubular member 102 may comprise a deformable material such that the tubular member 102 may be deformed to seal the passageway 102A or released to unseal the passageway 102A.

By way of example and without limitation, the tubular member 102 may comprise various polymers, rubbers, or other materials that are both deformable and resilient such that, absent application of force, the tubular member 102 reverts back to a shape in which the passageway 102A is not obstructed (i.e., unsealed). Put differently, the tubular member 102 may comprise a shape memory material so as to have a shape memory such that, when unconstrained (e.g., by cams 105A, 105B as described herein), the tubular member 102 reverts back to a tubular shape with its passageway 102A being unsealed.

In an example embodiment, the tubular member 102 may function as a gasket to seal or unseal the passageway 102A. The tubular member 102 may be deformed to seal the passageway 102A, such as by application of force (e.g., a pinching force) by one or more cams 105A, 105B as discussed herein. When released, the tubular member 102 will preferably revert back at least partially to its original shape in which the passageway 102A is unsealed.

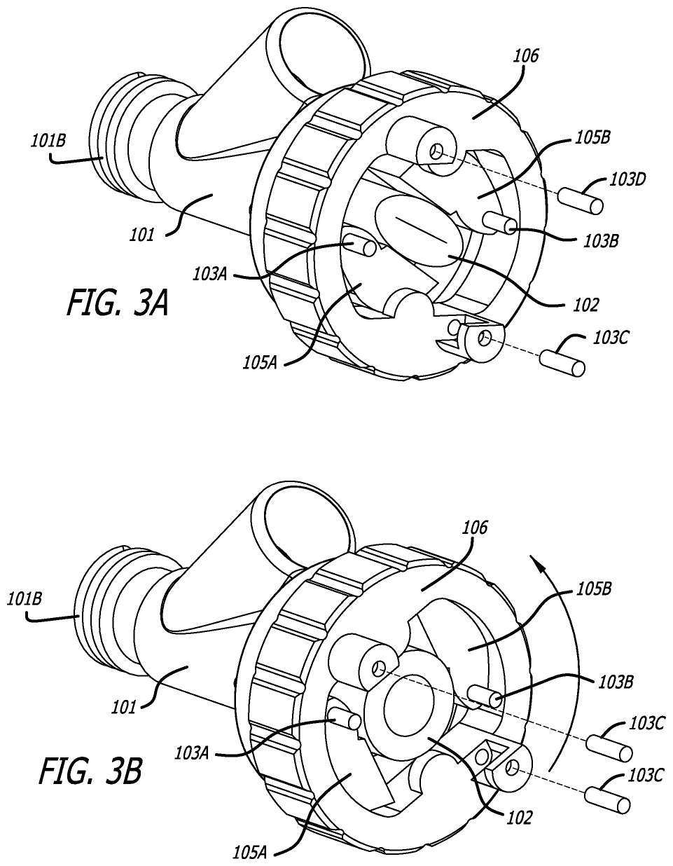

FIG. 3A-4B and 5A-5B illustrate a pair of cams 105A, 105B being positioned on either side of the tubular member 102. The cams 105A, 105B may be generally operable to deform (e.g., by pinching or squeezing) the tubular member 102 to seal the passageway 102A. The shape, size, orientation, positioning, and number of cams 105A, 105B may vary in different embodiments, and thus should not be construed as limited to the particular configuration shown in the exemplary figures.

In an example embodiment, the cams 105A, 105B may positioned on opposite sides of the tubular member 102. For example, in an embodiment in which the tubular member 102 is cylindrical, the cams 105A, 105B may be separated by 180 degrees along the outer circumference of the tubular member 102. However, it should be appreciated that various other positions may be utilized (e.g., the cams 105A, 105B may in some embodiments be positioned adjacent to each other).

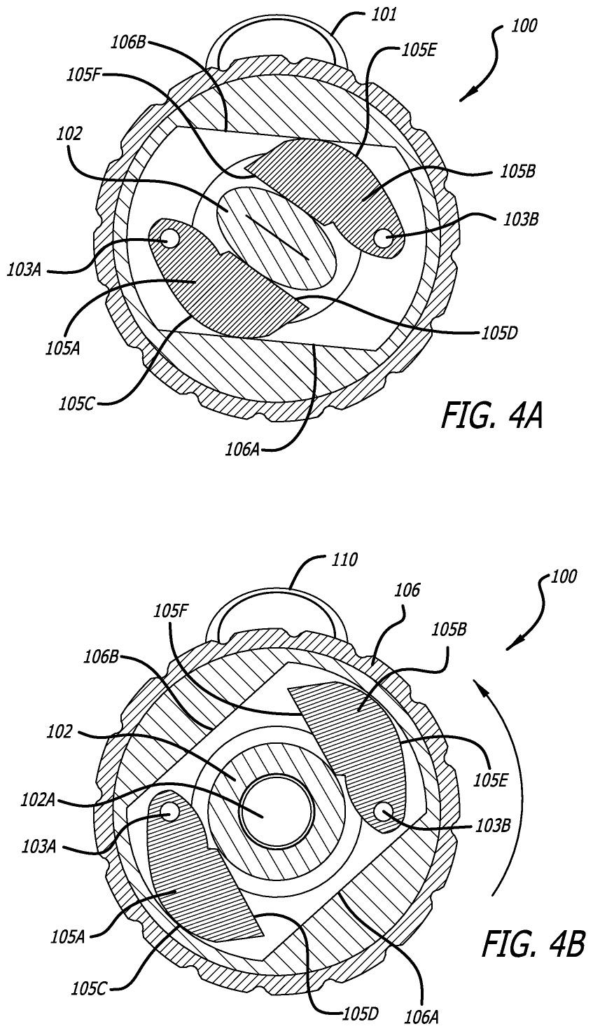

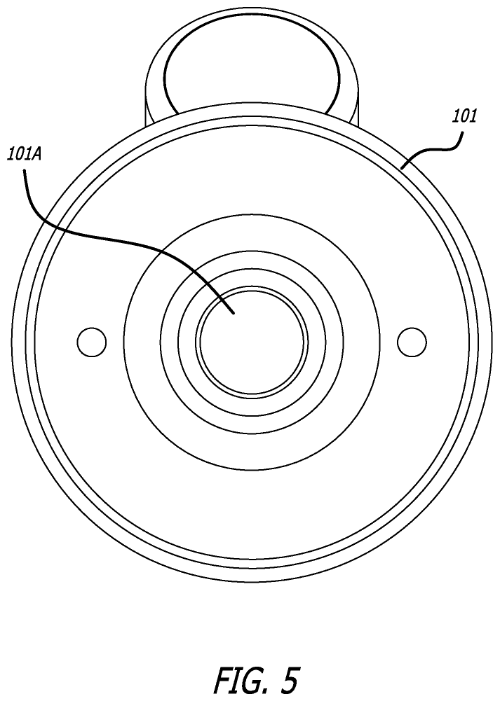

The cams 105A, 105B can best be seen in FIGS. 4A and 4B. As shown, the cams 105A, 105B may in an example embodiment each comprise the same shape and size, but opposite orientations. In some embodiments, however, the cams 105A, 105B may have different shapes or sizes with respect to each other. Each cam 105A, 105B may include an opening through which a pin 103A, 103B may extend as discussed in more detail below. The cams 105A, 105B may move or pivot about the pins 103A, 103B, or both the cams 105A, 105B and the pins 103A, 103B may rotate together relative to the remaining portions of the valve 100, in different embodiments.

Continuing to reference FIGS. 4A and 4B, each of the cams 105A, 105B may include curved outer surface and a flat inner surface. Thus, the first cam 105A may include a first curved outer surface 105C and a first flat inner surface 105D and the second cam 105B may include a second curved outer surface 105E and a second flat inner surface 105F. However, it should be appreciated that the inner surface(s) of the cams 105A, 105B may be convex, concave, or curved in some embodiments.

In embodiments with flat inner surfaces, the flat inner surfaces 105D, 105F of the respective cams 105A, 105B may abut against the tubular member 102 and, when engaged, deform the tubular member 102 to seal the passageway 102A. The inner edges of the actuator 106, which is discussed in more detail below, may include flat surfaces 106A, 106B which aid in forcing the cams 105A, 105B towards each other and maintaining the seal absent application of force. As shown in FIG. 4A, for example, the first flat surface 106A of the actuator 106 may engage with the first curved outer surface 105C of the first cam 105A and the second flat surface 106B of the actuator 106 may engage with the second curved outer surface 105E of the second cam 105B.

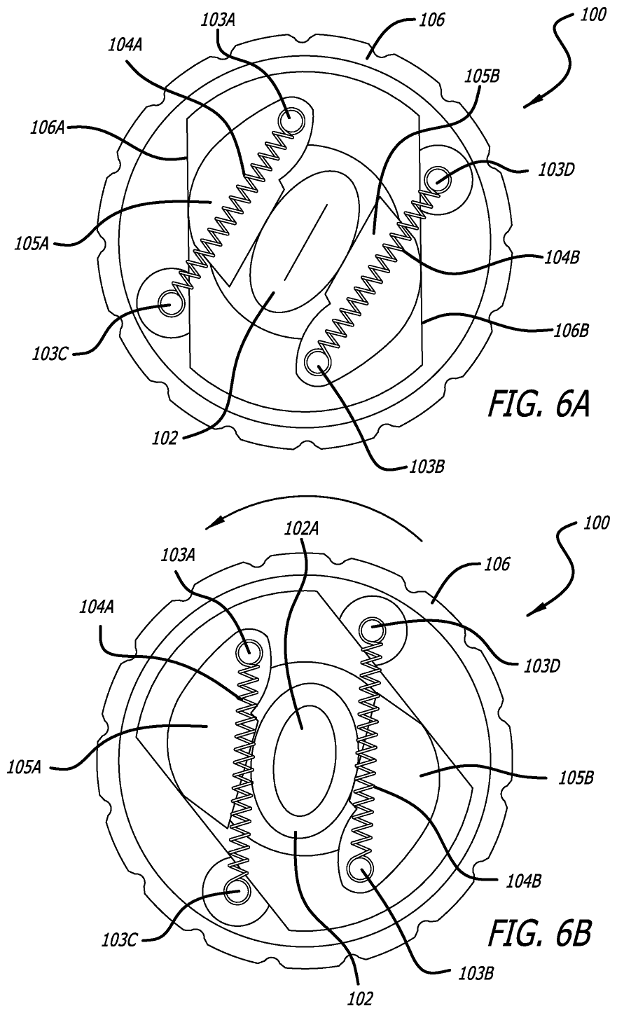

As best shown in FIGS. 4A and 5A, the cams 105A, 105B together may function as a vice between which the tubular member 102 is positioned, with the flat surfaces 105D, 105F of the cams 105A, 105B being brought closer together to seal the passageway 102A as shown in FIGS. 4A and 6A and brought further apart to unseal the passageway 102A as shown in FIGS. 4B and 6B.

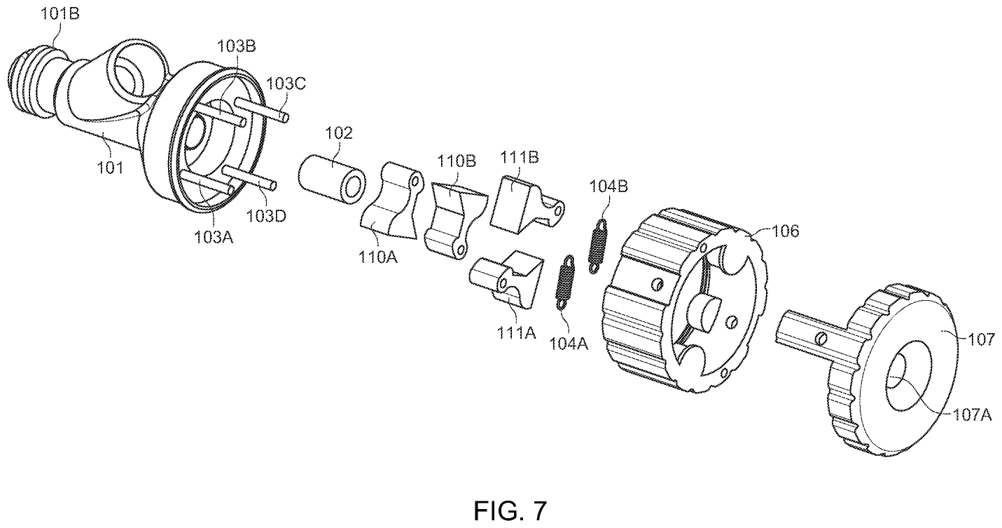

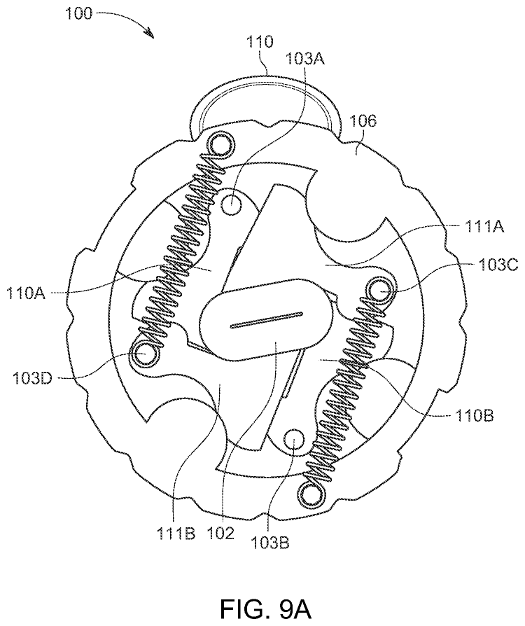

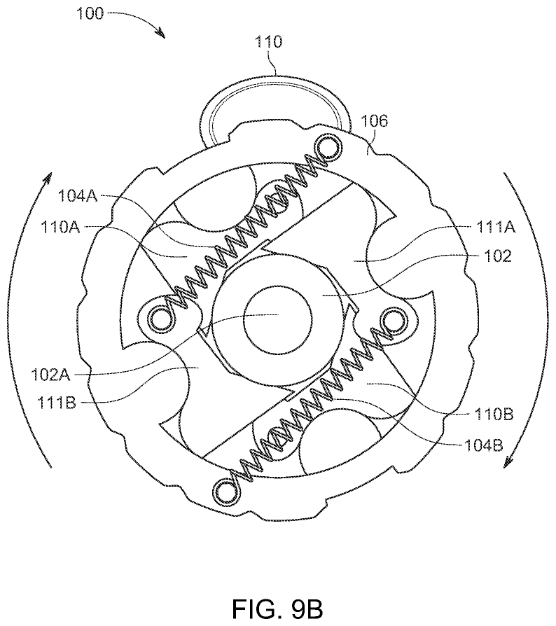

It should be appreciated that the number of cams 105A, 105B may vary in different embodiments. FIGS. 2A-4B and 6A-6B illustrate a pair of cams 105A, 105B comprising a first cam 105A and a second cam 105B. However, in some embodiments, only a single cam 105A may be utilized. In other embodiments, three or more cams 105A, 105B may be utilized. FIGS. 7-9B illustrate a first pair of cams 110A, 110B comprising a first cam 110A and a second cam 110B and a second pair of cams 111A, 111B comprising a third cam 111A and a fourth cam 111B.

The cams 105A, 105B may generally be operable to move between a first position in which the cams 105A, 105B apply sufficient force against the tubular member 102 to deform the tubular member 102 to seal the passageway 102A and a second position in which the cams 105A, 105B do not apply sufficient force against the tubular member 102 to seal the passageway 102A such that the passageway 102A is unsealed, opened, radially expanded, and/or unconstricted. When sealed, the passageway 102A will generally be sufficiently constricted, closed, shut, blocks, or clinched to prevent fluids (e.g., liquids and/or gasses) flowing through the passageway 102A.

The distance between the respective cams 105A, 105B when the passageway 102A is sealed will depend upon the diameter of the tubular member 102. When the passageway 102A is unsealed, the cams 105A, 105B may in some embodiments completely release (e.g., not contact) the tubular member 102 or, in other embodiments, may still contact the tubular member 102, but without sufficient force to deform sufficiently to seal. Thus, in some embodiments, the cams 105A, 105B may rest upon the outer surface of the tubular member 102 even when in the unsealed position. In some embodiments, the passageway 102A may be partially closed, but not completely sealed, when in the unsealed position.

The cams 105A, 105B may each comprise a distinct, independent structure such as shown in the figures. In such embodiments, the cams 105A, 105B may not be connected (e.g., directly connected) to, or in contact with, each other. In other embodiments, the cams 105A, 105B may be linked together in various manners.

The manner in which the cams 105A, 105B are adjusted between positions may vary in different embodiments. In the example embodiment shown in the figures, the cams 105A, 105B are illustrated as being pivotable between positions such that each cam 105A, 105B pivots between the sealed and the unsealed positions. However, in some embodiments, the cams 105A, 105B may be adjustable in various non-pivotable manners, e.g., by sliding or otherwise moving inwardly towards each other or outwardly away from each other.

In embodiments in which the cams 105A, 105B pivot between positions, the first cam 105A may pivot about a first pivot point and the second cam 105B may pivot about a second pivot point, with the first and second pivot points being distally spaced with respect to each other. Further, the first cam 105A may pivot in a first direction and the second cam 105B may pivot in a second direction, with the first direction being opposite to the second direction.

Continuing to reference embodiments in which the cams 105A, 105B pivot between positions, each of the cams 105A, 105B may be connected to a pin 103A, 103B. Thus, the first cam 105A may be connected to a first pin 103A and the second cam 105B may be connected to a second pin 103B. In embodiments in which additional cams 105A, 105b are utilized, additional pins 103A, 103B may also be utilized. Accordingly, it should be appreciated that, while the figures illustrate only a pair of pins 103A, 103B, three or more pins 103A, 103B may be utilized in some embodiments.

Further, in an example embodiment, only one pin 103A may be utilized (e.g., in an embodiment in which only a single cam 105A is utilized or in an embodiment in which multiple cams 105A, 105B are connected to a single pin 103A such as embodiments in which multiple cams 105A, 105B at least partially overlap each other). Thus, only one pin 103A may be utilized in embodiments in which only a single cam 105A is used, and three or more pins 103A, 103B may be utilized in embodiments in which three or more cams 105A, 105B are used.

The pins 103A, 103B may function as pivot members which function to pivot the cams 105A, 105B. The cams 105A, 105B may each pivot about a respective pin 103A, 103B, or, in other embodiments, the cams 105A, 105B may be fixed to each pin 103A, 103B such that the cams 105A, 105B pivot with a respective pin 103A, 103B. Each pin 103A, 103B may thus comprise an elongated member such as a rod or the like. The pins 103A, 103B may comprise various types of materials, such as but not limited to metals, alloys, polymers, and the like.

In the embodiment shown in FIGS. 4A, 4B, 6A, and 6B, it can be seen that in an example embodiment one or more pins 103A, 103B may be positioned on either side of the tubular member 102. In the example embodiment shown in the figures, a first pin 103A is shown positioned along a first side of the tubular member 102 and a second pin 103B is shown positioned along a second side of the tubular member 102. in an embodiment in which the tubular member 102 is cylindrical, the pins 103A, 103B may be separated by 180 degrees along the outer circumference of the tubular member 102. However, it should be appreciated that various other positions may be utilized (e.g., the pins 103A, 103B may in some embodiments be positioned adjacent to each other).

In the embodiment best shown in FIGS. 4A, 4B, 6A, and 6B, it can be seen that each pin 103A, 103B may extend through a corresponding cam 105A, 105B. Thus, each of the cams 105A, 105B may include an opening from which a pin 103A, 103B may extend. As previously mentioned, each cam 105A, 105B may be fixed to a pin 103A, 103B such that the cam 105A, 105B pivots with the pin 103A, 103B, or each cam 105A, 105B may be pivotably connected to a pin 103A, 103B such that the cam 105A, 105B pivots about the pin 103A, 103B.

Each pin 103A, 103B may be attached or fixed to the housing 101 such as shown in FIGS. 2A and 2B. In such embodiments, each pin 103A, 103B may be fixed within an opening in the housing 101. The manner by which the pins 103A, 103B are secured to the housing 101 may vary in different embodiments. By way of example, the pins 103A, 103B may be secured to the housing 101 by frictional engagement, adhesives, or the like. The pins 103A, 103B may be removable from the housing 101 in some embodiments. The pins 103A, 103B may each be rotatable within such openings in the housing 101 in embodiments in which the cams 105A, 105B are fixed to the pins 103A, 103B, or may be fixed within such openings in the housing 101 in embodiments in which the cams 105A, 105B pivot about the pins 103A, 103B.

The cams 105A, 105B may be biased towards the closed, or sealed position such that, absent application of force, the cams 105A, 105B seal the passageway 102A. Because the cams 105A, 105B are biased towards sealing the passageway 102A, an operator such as a physician can be assured that, absent an active application of force, the passageway 102A is sealed. This can aid in preventing errors in which an operator may think that the passageway 102A has been sealed when it hasn't (e.g., such as with valves which are not biased).

The manner by which the cams 105A, 105B are biased may vary in different embodiments. In an example embodiment, the cams 105A, 105B may be biased by one or more biasing members 104A, 104B. In one example embodiment, a single biasing member 104A, 104B may bias multiple cams 105A, 105B, such as a pair of cams 105A, 105B, by itself. In other embodiments, each cam 105A, 105B may be separately biased by one or more biasing members 104A, 104B.

In the example embodiments shown in the figures, a first cam 105A may be biased by a first biasing member 104A and a second cam 105B may be biased by a second biasing member 104B. Various types of biasing members 104A, 104B may be used in different embodiments. In the example embodiment shown in the figures, each of the biasing members 104A, 104B may comprise a spring. Various types of springs may be utilized, such as compression springs, extension springs, torsion springs, constant force springs, and the like. The figures illustrate an embodiment in which each biasing member 104A, 104B may comprise a coil spring.

The one or more biasing members 104A, 104B may be attached at a first end to a cam 105A, 105B and at a second end to an actuator 106. It should be appreciated, however, that in some embodiments the biasing members 104A, 104B may be attached to various other components. In some example embodiments, a first end of each biasing member 104A, 104B may be attached to a corresponding pin 103A, 103B and a second end of each biasing member 104A, 104B may be attached to the actuator 106.

In the embodiment shown in FIGS. 6A and 6B, it can be seen that a first end of each biasing member 104A, 104B may include an eyelet having an opening through which a pin 103A, 103B extends. The opposite, second end of each biasing member 104A, 104B may optionally also include an eyelet having an opening for attaching (e.g., fixedly) to the actuator 106.

As best shown in FIGS. 3A, 3B, 6A, and 6B, additional pins 103C, 103D may be connected to the actuator 106 at a first end and to a biasing member 104A, 104B at a second end. More specifically, it can be seen that each of a pair of pins 103C, 103D may be secured within a receiver formed in the actuator 106 body such as best shown in FIGS. 3A and 3B. The second pair of pins 103C, 103D may be the same size, or may be a different size, than the first pair of pins 103A, 103B.

Each of the second pair of pins 103C, 103D may function to anchor one of the biasing members 104A, 104B to the actuator 106. In the example embodiment shown in the figures, it can be seen that a third pin 103C may anchor the first biasing member 104A to the actuator 106 at a first radial location and that a fourth pin 103D may anchor the second biasing member 104B to the actuator 106 at a second radial location.

Thus, in the example embodiment shown in the figures, the first biasing member 104A may be secured at a first end to the first cam 105A by the first pin 103A and at a second end to the actuator 106 by the third pin 103C. Similarly, the second biasing member 104B may be secured at a first end to the second cam 105B by the second pin 103B and at a second end to the actuator 106 by the fourth pin 103D.

As each cam 105A, 105B is moved towards the opened or unsealed position, such as by activation (e.g., by rotation) of an actuator 106, each of the biasing members 104A, 104B will stretch or elongate such as shown in FIGS. 6A and 6B. Upon the actuator 106 being released, each biasing member 104A, 104B will naturally compress to its original state, thus adjusting the cams 105A, 105B back to their resting positions in which they seal the tubular member 102.

Various types of actuators 106 may be utilized to unseal the passageway 102A. In the example embodiment best shown in the figures, an actuator 106 is illustrated which may comprise a circular ring member which is configured to be rotated to adjust the cams 105A, 105B and thus unseal the passageway 102A. However, it should be appreciated that the shape, size, and configuration of the actuator 106 may vary in different embodiments. Thus, the scope of the present invention should not be construed as being limited to a circular ring-shaped actuator 106 as shown in the exemplary figures.

The illustrated example embodiment of an actuator 106 may include an outer edge (e.g., an outer circumference) and an inner edge (e.g., an inner circumference) defining a central opening. The outer edge of the actuator 106 may comprise grooves, ribbing, projections, or the like for improved grip. The inner edge of the actuator 106 may comprise one or more flat surfaces 106A, 106B for engaging with the cams 105A, 105B when in the sealed position as shown in FIGS. 4A and 6A. More specifically, it can be seen that the first flat surface 106A of the inner edge of the central opening of the actuator 106 may engage with the first cam 105A and that the second flat surface 106B of the inner edge of the central opening of the actuator 106 may engage with the second cam 105B when in the sealed position. In some embodiments, the inner edge of the actuator 106 may include one or more flanges to aid in gripping and engaging with the cams 105A, 105B.

In some embodiments, the actuator 106 may not comprise a circular ring member or may include additional features connected to a circular ring member. In such embodiments, the actuator 106 may comprise or further comprise, e.g., one or more levers, one or more buttons, or the like. For example, a handle, lever, button, actuator, or the like may be connected to or integrally formed with the ring member to aid in adjusting the ring member. The actuator 106 may comprise shapes other than the circular shape shown in the figures. By way of example, the actuator 106 may be square-shaped in some embodiments.

The manner by which the actuator 106 is adjusted to unseal the passageway 102A may vary in different embodiments. In the embodiment shown in the figures, it can be seen that the actuator 106 rotate in a first direction to unseal the passageway 102A and in a second direction to seal the passageway 102A. More specifically, it can be seen that the actuator 106 may rotate in a counterclockwise direction to unseal the passageway 102A and in a clockwise direction to seal the passageway 102A.

However, it should be appreciated that, in some embodiments, a clockwise rotation may instead be used to unseal the passageway 102A and a counterclockwise rotation may instead be used to seal the passageway 102A. In either case, the actuator 106 may be actively rotated to unseal the passageway 102A and, when released, may passively (e.g., without any input or force) revert to its original position, e.g., through action of the biasing member(s) 104A, 104B to seal the passageway 102A.

With reference to FIGS. 1-2B, it can be seen that a cap 107 may be connected to an end of the actuator 106. The cap 107 may function to enclose the tubular member 102, pins 103A, 103B, biasing members 104A, 104B, and cams 105A, 105B so as to prevent incursion of particulates such as dust or the like that may compromise operation of the biasing members 104A, 104B or cams 105A, 105B and/or prevent contact of these parts with fluids such as blood or other bodily fluids. Although the figures illustrate an embodiment in which the cap 107 is circular, it should be appreciated that the cap 107 may comprise various other shapes in certain embodiments.

The cap 107 may be removably attached to the actuator 106 such that the cap 107 may be removed to access the interior of the hemostatic valve 100, or the cap 107 may be fixed to the actuator 106. The cap 107 may include a cap inlet 107A, such as a central opening as shown in the figures, by which various medical devices may be inserted through or fluidly connected to the passageway 102A when unsealed. In an exemplary embodiment, the pins 103A, 103B may be secured to the cap 107. In such embodiments, the cap 107 may include openings through which the pins 103A, 103B extend or in which the pins 103A, 103B are attached.

FIGS. 7-9B illustrate an example embodiment of a hemostatic valve 100 which includes two pairs of cams 110A, 110B, 111A, 111B. A first pair of cams 110A, 110B, comprised of a first cam 110A and a second cam 110B, may be aligned along a first radial plane. A second pair of cams 111A, 111B, comprised of a third cam 111A and a fourth cam 111B, may be aligned along a second radial plane. Both of the first and second radial planes may be perpendicular to an axis of rotation of the cams 110A, 110B, 111A, 111B.

As shown in FIG. 7, the cams 110A, 110B, 111A, 111B may be connected to a housing 101 by one or more pins 103A, 103B, 103C, 103D. Each of the pins 103A, 103B, 103C, 103D may be attached to the housing 101. In some example embodiments, the housing 101 may include a number of openings corresponding to the number of pins 103A, 103B, 103C, 103D such that each pin 103A, 103B, 103C, 103D may be seated or fixed within its own opening.

The openings, and thus the pins 103A, 103B, 103C, 103D when connected, may be positioned at preset intervals from each other such that each of the pins 103A, 103B, 103C, 103D is at an equal distance from the remaining pins 103A, 103B, 103C, 103D. As shown in FIG. 7, a first cam 110A may be attached to pivot about a first pin 103A, a second cam 110B may be attached to pivot about a second pin 103B, a third cam 111A may be attached to pivot about a third pin 103C, and a fourth cam 111B may be attached to pivot about a fourth pin 103D.

Alternatively, in other example embodiments, the pins 103A, 103B, 103C, 103D may themselves be rotatably connected to the housing 101 such that each pin 103A, 103B, 103C, 103D may rotate with respect to the housing 101. In such example embodiments, the cams 110A, 110B, 111A, 111B may be fixed to their respective pins 103A, 103B, 103C, 103D such that each of the cams 110A, 110B, 111A, 111B pivots when its respective pin 103A, 103B, 103C, 103D rotates.

As previously mentioned, although the figures illustrate an example embodiment in which a pin 103A, 103B, 103C, 103D is provided for each cam 110A, 110B, 111A, 111B, in some example embodiments, multiple cams 110A, 110B, 111A, 111B may share a pin 103A, 103B, 103C, 103D.

Continuing to reference FIG. 7, it can be seen that a pair of biasing members 104A, 104B, such as springs, may be utilized to bias each of the cams 110A, 110B, 111A, 111B towards a desired position. In the example embodiment shown in FIG. 7, it can be seen that a pair of biasing members 104A, 104B may function to bias all of four cams 110A, 110B, 111A, 111B. In other example embodiments, each cam 10A, 110B, 111A, 111B may have its own biasing member 104A, 104B.

Each of the biasing members 104A, 104B may be connected at a first end to one of the pins 103A, 103B, 103C, 103D. In the example embodiment shown in FIGS. 7-9B, it can be seen that a first biasing member 104A may be attached to a first pin 103A and a second biasing member 104B may be attached to a second pin 103B. However, the number, positioning, and orientation of the biasing members 104A, 104B may vary in different embodiments.

A second end of each of the biasing members 104A, 104B may be attached to an actuator 106 as previously discussed. Rotation of the actuator 106 may be operable to pivot the cams 110A, 110B, 111A, 111B as discussed herein. Generally, rotation of the actuator 106 in a first direction may cause all or some of the cams 110A, 110B, 111A, 111B to pivot in a first direction, and rotation of the actuator 106 in a second direction may cause all or some of the cams 110A, 110B, 111A, 111B to pivot in a second direction.

A cap 107 may be attached to the actuator 106 as previously described. In the example embodiment shown in FIG. 7, it can be seen that the cap 107 may comprise one or more projections which may engage with corresponding indentations, such as grooves or slits, in the outer circumference of the actuator 106 so as to couple the cap 107 to the actuator 106 and thereby substantially enclose the cams 110A, 110B, 111A, 111B.

The cams 110A, 110B, 111A, 111B may be positioned radially about a tubular member 102 such as a seal such that, when engaged, the cams 110A, 110B, 111A, 111B deform, such as by pinching, the tubular member 102 and thereby seal a passageway 102A extending through the tubular member 102. The use of four cams 110A, 110B, 111A, 111B may more effectively seal the tubular member 102 than example embodiments in which less cams are utilized and, further, may provide redundancies in the event that one or more of the cams 110A, 110B, 111A, 111B fail to function.

As shown in FIGS. 7-9B, the actuator 106 may include one or more projections 115A, 115B, 115C, 115D which may function to guide and/or force the cams 110A, 110B, 111A, 111B between their respective pivotable positions. Each of the projections 115A, 115B, 115C, 115D may comprise a semi-circular nub or the like which extends inwardly from the actuator 106 such as shown in the figures.

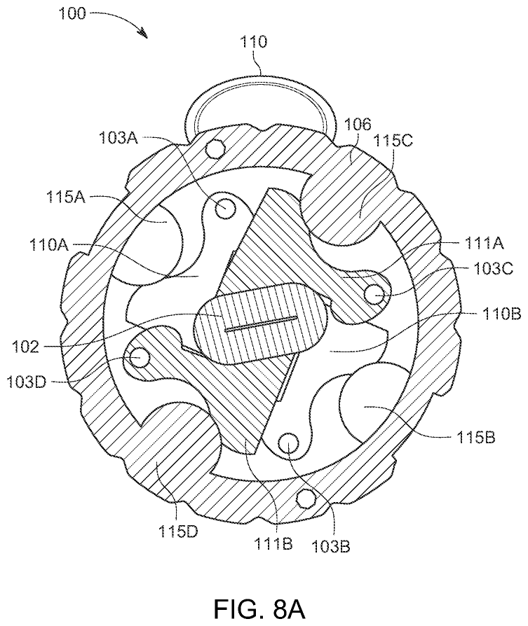

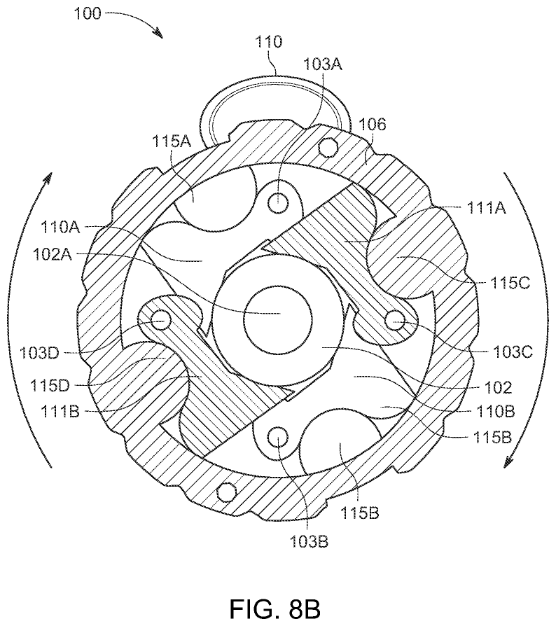

The number, positioning, spacing, and orientation of the projections 115A, 115B, 115C, 115D may vary in different embodiments. Generally, the projections 115A, 115B, 115C, 115D may be equally-spaced radially about the inner diameter of the actuator 106 such as best shown in FIGS. 8A-8B.

In the example embodiment shown in FIGS. 7-9B, it can be seen that a first projection 115A may engage with a first cam 110A, a second projection 115B may engage with a second cam 110B, a third projection 115C may engage with a third cam 111A, and a fourth projection 115D may engage with a fourth cam 111B.

In some example embodiments, the number of projections 115A, 115B, 115C, 115D may be the same as the number of cams 110A, 110B, 111A, 111B such that each cam 110A, 110B, 111A, 111B may be actuated by its own separate projection 115A, 115B, 115C, 115D. In other embodiments, two or more cams 110A, 110B, 111A, 111B may share a projection 115A, 115B, 115C, 115D such that there are less projections 115A, 115B, 115C, 115D than cams 110A, 110B, 111A, 111B.

Each of the cams 110A, 110B, 111A, 111B may include an inward curved portion on its outer edge into which the respective projections 115A, 115B, 115C, 115D may engage so as to adjust the cams 110A, 110B, 111A, 111B. However, other configurations and shapes may be utilized in different embodiments, so long as rotational movement of the actuator 106 imparts sufficient force to cause pivotable movement of the cams 110A, 110B, 111A, 111B.

In use, a catheter 110 or other access device may first be attached to a distal end of the housing 101 in embodiments in which a catheter 110 or access device is not integral with or previously fixed to the housing 101. The catheter 110 may then be inserted into the body of a patient by an operator and routed to a desired location to perform its function (e.g., aspiration). Alternatively, these steps may be reversed by first inserting the access device such as a catheter 110 into the body of the patient and routing to a desired location and then attaching the access device such as a catheter 110 to the housing 101.

Absent any application of force, the passageway 102A will remain sealed as the catheter 110 or other access device is routed through the body to its desired location. Thus, the operator may rest assured that there will not be any incursion of fluids, air, or the like into the body except when desired. Upon the catheter 110 or other access device reaching its target location, the hemostasis valve 100 may be operated.

FIGS. 3A, 4A, 6A, 8A, and 9A illustrate example embodiments of a hemostasis valve 100 in a sealed position and FIGS. 3B, 4B, 6B, 8B, and 9B illustrate example embodiments of a hemostasis valve 100 in an unsealed position. In one example embodiment, the hemostasis valve 100 may be unsealed by rotating the actuator 106. However, it should be appreciated that, as previously mentioned, various other types of actuators 106 may be utilized such that movement other than rotational movement may be used to unseal the hemostasis valve 100. For example, in some embodiments, the actuator 106 may instead be squeezed to unseal the hemostasis valve 100.

Continuing to reference the embodiment shown in the figures, it can be seen that the actuator 106 may be rotated in a first direction to at least partially release the cams 105A, 105B from the tubular member 102 so as to unseal the passageway 102A. As the actuator 106 is rotated in the first direction, the attached biasing members 104A, 104B may be pulled upon by the actuator 106 such that the biasing members 104A, 104B both stretch and adjust the cams 105A, 105B towards the unsealed position.

FIGS. 4A and 4B illustrate an example embodiment in which the first direction which unseals the passageway 102A may comprise a counterclockwise direction. FIGS. 6A and 6B illustrate another example embodiment in which the first direction which unseals the passageway 102A may comprise a clockwise direction. Thus, it should be appreciated that the direction in which the actuator 106 is adjusted to open or close the passageway 102A may vary in different embodiments.

In the example embodiment shown in FIGS. 1-6B, it can be seen that the biasing members 104A, 104B may be attached to the pins 103A, 103B; with the cams 105A, 105B being attached to the pins 103A, 103B. More specifically, the first biasing member 104A may be attached to the first pin 103A which is attached to the first cam 105A and the second biasing member 104B may be attached to the second pin 103B which is attached to the second cam 105B. Thus, in such an embodiment, the biasing members 104A, 104B may pull on the pins 103A, 103B which function to move the cams 105A, 105B away from the tubular member 102 to unseal the passageway 102A. Additionally or alternatively, the flange of the actuator 106 may force the cams 105A, 105B to move away from the tubular member 102 to unseal the passageway 102A.

In the example embodiment shown in FIGS. 7-9B, it can be seen that each of the four cams 110A, 110B, 111A, 111B may be connected to its own separate pin 103A, 103B, 103C, 103D, but that only pins 103A and 103B are connected to biasing members 104A, 104B, respectively. As previously mentioned, in some embodiments, each cam 110A, 110B, 111A, 111B may have its own separate spring. However, the example embodiment shown in FIGS. 7-9B instead relies upon only a pair of biasing members 104A, 104B to provide the biasing force towards the sealed or closed position, and separately relies upon the aforementioned projections 115A, 115B, 115C, 115D to aid in forcing the cams 110A, 110B, 111A, 111B between their respective positions.

The amount (e.g., degree) of rotational movement of the actuator 106 necessary to unseal the passageway 102A may vary in different embodiments. Preferably, minimal rotational movement will be needed such that minimal effort is required by the operator to unseal the passageway 102A. By way of example, the actuator 106 may be rotated approximately 45 degrees in a first direction to unseal the passageway 102A. However, in some embodiments, less than 45 degrees rotational movement may be used to unseal the passageway 102A. In yet other embodiments, more than 45 degrees rotational movement (e.g., 60 degrees, 90 degrees, 120 degrees, 180 degrees, or more) may be used to unseal the passageway 102A.

Preferably, constant force (e.g., rotational force) will need to be applied to the actuator 106 to keep the cams 105A, 105B, 110A, 110B, 111A, 111B from reverting back to their original position sealing the passageway 102A. Such a configuration ensures that the passageway 102A is never unsealed except when desired. In such a manner, the operator can be assured that the passageway 102A is always sealed except when the actuator 106 is manually adjusted to unseal the passageway 102A. This prevents mistakes which can occur in valves that are not biased towards the sealed position (e.g., situations in which an operator may forget to manually adjust the actuator 106 back to seal the passageway 102A).

With the passageway 102A unsealed, the operator may advance a desired access device through the passageway 102A, such as but not limited to a guidewire, an implant delivery catheter, a balloon catheter, an aspiration catheter, a clot retrieval catheter, or any type of known catheter or intravascular medical device. delivery catheter, a balloon catheter, an aspiration catheter, a clot retrieval catheter, any type of known catheter or intravascular medical device.

For example, various medical devices may be inserted through the cap inlet 107A, the unsealed passageway 102A, and the housing lumen 101A to enter the catheter 110 and be advanced to a desired position within the patient's vasculature. Upon achieving a desired position with the device, the operator may release the actuator 106. While a medical device is inserted to extend through the passageway 102A of the valve 100, the biasing force from the biasing members 104A, 104B may force the tubular member 102 into contact with the medical device such that the passageway 102A seals around the medical device. Thus, when a medical device is inserted therethrough, the valve 100 may take on a third position in which the tubular member 102 is deformed around the medical device. Generally, this third position will be between the positions previously referred to as unsealed and sealed positions.

Upon completion of use of the medical device, the operator may again adjust the actuator 106, such as by rotation, to release the tubular member 102 from around the medical device and unseal the passageway 102A such that the medical device may be removed therefrom. The operator may then release the actuator 106, at which time the biasing members 104A, 104B will naturally revert the cams 105A, 105B, 110A, 110B, 111A, 111B and actuator 106 back to their original, sealed position in which the passageway 102A is sealed. The same steps may be repeated as needed during the medical procedure.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1-26. (canceled)

27. A hemostasis valve, comprising:
a tubular member including a passageway;
at least one cam, wherein the at least one cam is adjustable between a first position in which the at least one cam deforms the tubular member to seal the passageway and a second position in which the at least one cam releases the tubular member to at least partially unseal the passageway; and
an actuator connected to the at least one cam for adjusting the at least one cam between the first position and the second position.

28. The hemostasis valve of claim 27, wherein the at least one cam comprises a first cam and a second cam, and wherein the first cam is not directly connected to the second cam.

29. The hemostasis valve of claim 28, wherein the first cam pivots about a first pivot point in a first direction and wherein the second cam pivots about a second pivot point in a second direction, wherein the first direction is opposite to the first direction.

30. The hemostasis valve of claim 29, wherein the tubular member is pinched between the first cam and the second cam when the first cam and the second cam are in the first position.

31. The hemostasis valve of claim 28, further comprising a first biasing member connected to the first cam and a second biasing member connected to the second cam.

32. The hemostasis valve of claim 31, wherein the first biasing member is connected between the first cam and the actuator, and wherein the second biasing member is connected between the second cam and the actuator.

33. The hemostasis valve of claim 31, wherein the first biasing member comprises a first spring and wherein the second biasing member comprises a second spring.

34. The hemostasis valve of claim 27, wherein the tubular member further comprises a gasket.

35. The hemostasis valve of claim 27, wherein the actuator comprises a ring member including a flange for engaging with the at least one cam.

36. The hemostasis valve of claim 27, wherein the actuator is rotatable to adjust the at least one cam between the first position and the second position.

37. The hemostasis valve of claim 36, wherein the at least one cam is pivotable between the first position and the second position.

38. The hemostasis valve of claim 27, further comprising one or more pins, and wherein the at least one cam is pivotably connected to the one or more pins.

39. The hemostasis valve of claim 27, further comprising a housing including an internal lumen for connecting to a catheter, wherein the tubular member is connected to the housing, and wherein the passageway is fluidly connected to the internal lumen.

40. The hemostasis valve of claim 39, further comprising one or more pins connected to the housing, and wherein the at least one cam is connected to the one or more pins.

41. The hemostasis valve of claim 27, wherein the at least one cam comprises a first pair of cams including a first cam and a second cam and a second pair of cams including a third cam and a fourth cam.

42. The hemostasis valve of claim 41, wherein the first cam and the second cam are aligned along a first radial plane and wherein the third cam and the fourth cam are aligned along a second radial plane.

43. The hemostasis valve of claim 42, wherein the first radial plane is positioned outwardly with respect to the second radial plane.

44. The hemostasis valve of claim 27, wherein each of the at least one cam comprises a curved outer surface, and further comprising a plurality of projections extending inwardly from the actuator for engaging with the curved outer surface of the at least one cam.

45. The hemostasis valve of claim 27, wherein the at least one cam is biased towards the first position.

46. A hemostasis valve, comprising:
a tubular member including a passageway;
a sealing means for closing the passageway, the sealing means being adjustable between a first position in which the sealing means deforms the tubular member to seal the passageway and a second position in which the sealing means releases the tubular member to at least partially unseal the passageway; and
an actuation means for adjusting the sealing means between the first position and the second position, the actuation means being connected to the sealing means; and,
a biasing means for biasing the sealing means towards the first position.
